(19) United States
(12) Patent Application Publication
(10) Pub. No.: US 2026/0240862 A1
(43) Pub. Date: Aug. 20, 2026

(54) COMBINATION OF ROGINOLISIB AND BCL-2 INHIBITOR IN THE TREATMENT OF HAEMATOLOGICAL MALIGNANCY

(71) Applicant: IONCTURA SA, Genève (CH)

(72) Inventors: Karolina NIEWOLA-STASZKOWSKA, Genève (CH); Michael LAHN, Genève (CH); Giusy DI CONZA, Genève (CH); Francesco BERTONI, Bellinzona (CH); Chiara TARANTELLI, Bellinzona (CH)

(21) Appl. No.: 19/161,838

(22) PCT Filed: Mar. 1, 2024
(86) PCT No.: PCT/EP2024/055392

§ 371 Date: Sep. 3, 2025

(30) Foreign Application Priority Data
Mar. 3, 2023 (GB) 2303190.9
May 31, 2023 (GB) 2308119.3

(51) Int. Cl. A61K 31/5377; A61K 31/519; A61K 31/635; A61K 45/06; A61P 35/00

(52) U.S. Cl. CPC A61K 31/5377; A61K 31/519; A61K 31/635; A61K 45/06; A61P 35/00

(57) ABSTRACT

A PI3K-delta inhibitor or a pharmaceutically acceptable salt thereof, for use in the treatment of a haematological malignancy in a subject, wherein said treatment comprises the separate, sequential or simultaneous administration of i) said PI3K-delta inhibitor, or a pharmaceutically acceptable salt thereof, and ii) a BCL-2 inhibitor, or a pharmaceutically acceptable salt thereof, to said subject. The treatment can also further comprise the separate, sequential or simultaneous administration of iii) a further chemotherapeutic agent, to said subject.

The present disclosure relates to methods of treating haematological malignancies in a patient in need thereof.

The application claims the benefit of priority to GB application No. 2303190.9, filed on 3 Mar. 2023, and GB application No. 2308119.3 filed on 31 May 2023, which are incorporated herein by reference in its entirety.

### BACKGROUND

Haematological malignancies, also known as blood cancers, are malignant neoplasms derived mostly from the myeloid and lymphoid cell lineages. Lymphoma, lymphocytic leukaemia, and myeloma are derived from the lymphoid lineage, while acute and chronic myelogenous leukemia, myelodysplastic syndromes and myeloproliferative diseases are of myeloid origin.

Haematological malignancies are a significant cause of morbidity and mortality worldwide, with almost 1,000,000 new cases and over 600,000 deaths annually (Jephcote 2020). Several agents contribute to the development of blood cancers, including occupational, lifestyle, and hereditary risk factors. Haematological malignancies are a heterogeneous disease for which outcomes can vary. Indolent lymphoma and chronic leukaemia, such as follicular lymphoma (FL), marginal zone lymphoma (MZL), chronic lymphocytic leukaemia (CLL) and small lymphocytic lymphoma (SLL), remain incurable chronic diseases, requiring patients to undergo repeated exposures to toxic therapies. For aggressive lymphoma and acute leukaemia, modern treatment regimens result in long-term survival rates ranging from >80% for Hodgkin lymphoma to ~60-65% for diffuse large B-cell lymphoma (DLBCL) and acute lymphoblastic leukaemia (ALL), and to <30% for peripheral T-cell lymphoma (PTCL) and acute myeloid leukaemia (AML) (Intlekofer and Younes 2014, Kantarjian 2021). More effective therapies are needed to improve outcome for patients with haematologic malignancies. Furthermore, even for those patients who are cured by combination chemotherapy, often used in conjunction with radiation, this therapy can result in long-term toxicities that impair health, predispose patients to secondary malignancies, and negatively impact quality of life.

Targeted therapies including phosphatidylinositol 3-kinase (PI3K) inhibitors and B-cell lymphoma 2 (BCL-2) inhibitors have changed the treatment landscape for patients with haematological malignancies. PI3Ks play a central role in the regulation of key intercellular pathways including growth, proliferation, survival, migration and differentiation, and dysregulation of the PI3K pathway is one of the most frequent pathogenic events in cancer (Kienle and Stilgenbauer 2020). BCL-2, a family of regulatory proteins, governs by the mitochondrial pathway of apoptosis and BCL-2 dysregulation commonly arises from genetic abnormalities, such as the t(14;18)(q32;q21) translocation in follicular lymphoma (FL) or focal deletion of chromosome 13 (del[13q14]) that leads to loss of negative regulatory miRNA-15a/16-1 of BCL-2 in CLL (Kapoor 2020). Thus, PI3K and BCL-2 are rational therapeutic targets in haematological malignancies.

Although PI3K and BCL-2 inhibitors have demonstrated impressive clinical outcomes across a variety of haematological malignancies, certain subtypes are characterized by significantly higher response rates than others and resistance develops to these inhibitors eventually leading to clinical disease progression. Also, both types of inhibitors come with severe adverse effects limiting their therapeutic applicability, and in the case of PI3K inhibitors even resulting in drug withdrawals from the market (Hampel 2021, Richardson 2022).

BCL-2 as a therapeutic target for haematological malignancies is discussed in Pereini 2018.

While much progress has been made in the treatment of haematological malignancies, there is increasing demand for targeted therapy for haematological malignancies. Many patients who have such cancers live with an incurable disease. Accordingly, it is important to continue to find new treatments for patients with incurable cancer that are more effective.

### SUMMARY

The present invention is directed to a new treatment for patients with haematological malignancies. In some embodiments, disclosed is a method of treating a haematological malignancy in a subject in need thereof, comprising administering to the subject a first amount of a compound of Formula I:

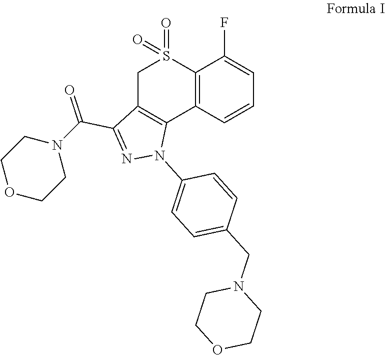 [SMILES: O=C(c1nn(-c2ccc(CN3CCOCC3)cc2)c2c1CS(=O)(=O)c1c(F)cccc1-2)N1CCOCC1]

or a pharmaceutically acceptable salt thereof, and a second amount of a BCL-2 inhibitor or a pharmaceutically acceptable salt thereof. In the method, the first amount and the second amount together comprise a therapeutically effective amount. The compound of Formula I may be referred to herein as “Compound 1”. In some of these embodiments, a third amount of a further chemotherapeutic agent is administered.

In some embodiments, disclosed is Compound 1, or a pharmaceutically acceptable salt thereof, for use in the treatment of a haematological malignancy in a subject, wherein said treatment comprises the separate, sequential or simultaneous administration of i) said Compound 1, or a pharmaceutically acceptable salt thereof, and ii) a BCL-2 inhibitor, or a pharmaceutically acceptable salt thereof, to said subject. In some of these embodiments, the treatment further comprises the separate, sequential or simultaneous administration of iii) a further chemotherapeutic agent, to said subject.

In some embodiments, disclosed is a BCL-2 inhibitor, or a pharmaceutically acceptable salt thereof, for use in the treatment of a haematological malignancy in a subject, wherein said treatment comprises the separate, sequential or simultaneous administration of i) said a BCL-2 inhibitor, or a pharmaceutically acceptable salt thereof, and ii) Compound 1, or a pharmaceutically acceptable salt thereof, to said subject. In some of these embodiments, the treatment further comprises the separate, sequential or simultaneous administration of iii) a further chemotherapeutic agent, to said subject.

In some embodiments, disclosed is the use of Compound 1, or a pharmaceutically acceptable salt thereof, in the manufacture of a medicament for use in the treatment of a haematological malignancy, wherein said treatment comprises the separate, sequential or simultaneous administration of i) said medicament comprising Compound 1, or a pharmaceutically acceptable salt thereof, and ii) a BCL-2 inhibitor, or a pharmaceutically acceptable salt thereof, to said subject. In some of these embodiments, the treatment further comprises the separate, sequential or simultaneous administration of iii) a further chemotherapeutic agent, to said subject.

In the above embodiments, the haematological malignancy may be lymphoma, leukaemia, myeloma, myelodysplastic syndrome and myeloproliferative disease.

In some embodiments, disclosed is a pharmaceutical product comprising i) Compound 1 or a pharmaceutically acceptable salt thereof, and ii) a BCL-2 inhibitor or a pharmaceutically acceptable salt thereof. In some of these embodiments, the pharmaceutical product further comprises iii) a further chemotherapeutic agent.

In some embodiments, disclosed is a kit comprising: a first pharmaceutical composition comprising Compound 1, or a pharmaceutically acceptable salt thereof; a second pharmaceutical composition comprising a BCL-2 inhibitor, or a pharmaceutically acceptable salt thereof; and instructions for using the first and second pharmaceutical compositions in combination. In some of these embodiments, the kit further comprises a third pharmaceutical composition comprising a further chemotherapeutic agent, and the instructions are for using the first, second and third pharmaceutical compositions in combination.

The combination of Compound 1 and a BCL-2 inhibitor (as well as the further chemotherapeutic agent, if present) may be synergistic, and/or may otherwise result in improved treatment outcomes or patient prognosis, for example the reduction of side effects and improved tolerability, when compared to current monotherapies or combination therapies. This may result from Compound 1 blocking survival pathways that are activated once BCL-2 inhibitors are given and vice versa.

### BRIEF DESCRIPTION OF THE DRAWINGS

FIG. 1 shows the dose response data of the combination of compound 1 and Venetoclax in the HH cell line, a cutaneous T-cell lymphoma cell line.

FIG. 2 shows the dose response data of the combination of compound 1 and Venetoclax in the SP53 cell line, a mantle cell lymphoma cell line.

### DETAILED DESCRIPTION

Compound 1 is example 339 in WO2011/058149, which document is incorporated herein by reference in its entirety. Its structure is according to Formula I:

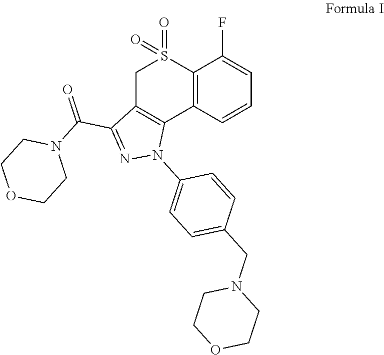 [SMILES: O=C(c1nn(-c2ccc(CN3CCOCC3)cc2)c2c1CS(=O)(=O)c1c(F)cccc1-2)N1CCOCC1]

In IUPAC nomenclature, the above Compound 1 may be referred to as 6-Fluoro-3-(morpholin-4-ylcarbonyl)-1-[4-(morpholin-4-ylmethyl)phenyl]-1,4-dihydrothiochromeno[4,3-c]pyrazole 5,5-dioxide. Alternatively, the structural formula shown above may be described as [6-fluoro-1-(4-morpholin-4-yl-methylphenyl)-5,5-dioxo-4,5-dihydro-1H-5λ6-thiochromeno[4,3-C]pyrazol-3-yl]-morpholin-4-yl-methanone.

Compound 1 can be prepared and characterized as described in published patent application WO 2011/058149 A1 (see compound 339 on p. 69; the preparation on p. 303-307; and the characterization on p. 481 with p. 414-418), which information is specifically incorporated herein by reference.

Based on the process disclosed in WO 2011/058149 A1, the authors of Haselmayer 2014 describe a five-step preparation procedure for the compound. This procedure starts with reaction of 8-fluoro-2,3-dihydro-4Hthiochromen-4-one with diethyl oxalate in the presence of sodium ethoxide. The intermediate is cyclized with 4-(4-hydrazinylbenzyl)morpholine to form a pyrazole ring. The thioether is then oxidized to the corresponding sulfone by reaction with meta-chloroperbenzoic acid, followed by saponification of the ethyl ester into the corresponding acid and subsequent coupling with morpholine to yield the compound of formula I.

Alternatively, the intermediate of the reaction of 8-fluoro-2,3-dihydro-4H-thiochromen-4-one with diethyl oxalate in the presence of sodium ethoxide is cyclized with 4-hydrazinobenzoic acid. The benzoic acid is reduced using borane-THF complex and the thioether is oxidized to the corresponding sulfone by reaction with meta-chloroperbenzoic acid. Saponification of the ethyl ester into the corresponding acid and chlorination of both the acid and alcohol with excess thionyl chloride in the presence of dimethylformamide and subsequent coupling with morpholine then yields Compound 1.

Compound 1 may be provided as a pharmaceutically acceptable salt. Suitable pharmaceutically acceptable salts are known in the art. Some pharmaceutically acceptable salts of Compound 1 are described in WO2014/121901, which is incorporated by reference in its entirety.

As used herein, Compound 1 is provided as an anhydrous hemifumarate salt (formula illustrated). Its synthesis and characterisation are described in WO2014/121901 (page 4). It is referred to as solid form A1. A hemifumarate hydrate (H1) has also been identified. The anhydrous hemifumarate salt used is crystalline and has a powder X-ray peak list as described in WO2014/121901. It will be appreciated that the findings of the invention are not limited to use of this solid form, although it is preferred.

Accordingly, in some cases Compound 1 is administered as the hemifumarate salt (Formula Ia). However, it will be understood that the invention is not so limited, and other solid forms (for example, other pharmaceutically acceptable salts) are envisaged.

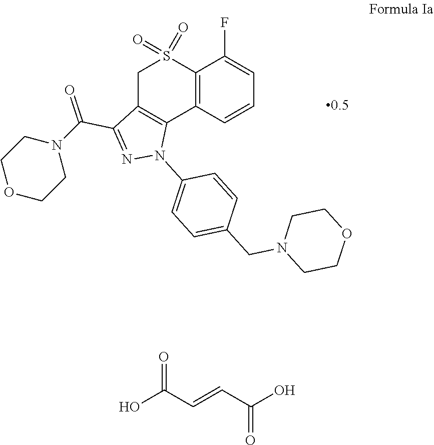 [SMILES: O=C(c1nn(-c2ccc(CN3CCOCC3)cc2)c2c1CS(=O)(=O)c1c(F)cccc1-2)N1CCOCC1 ; O=C(O)/C=C/C(=O)O]

Haselmayer 2014 also describes the characterisation of the compound as highly selective PI3Kδ inhibitor. Tarantelli 2022 further describes the activity of Compound 1 in lymphoma cell lines and Carlo-Stella 2022 reports the data on the first dose cohort of the clinical study of Compound 1 in FL patients (NCT04328844).

BCL-2 is a family of regulator proteins encoded by the BCL-2 gene that regulates cell death.

The BCL-2 family can be subclassified into different groups based on their morphology and Bcl-2 homology (BH) domain and consists of anti-apoptotic- and pro-apoptotic members (Ploumaki 2023). The term BCL-2 inhibitor includes targeted and selective inhibitors of anti-apoptotic protein members of the BCL-2 family such as BCL-2, BCL-W, BCL-XL, MCL-1, A1 and BFL-1.

BCL-2 inhibitors include but are not limited to Navitoclax, Obatoclax mesylate, Venetoclax, Lisaftoclax, LP-118, S55746, S64315, or Palcitoclax.

Venetoclax (also referred as ABT-199 and sold under the brand names Venclexta and Venclyxto) is an orally bioavailable BCL-2 inhibitor with high selectivity for BCL-2 protein and significantly lower affinity for BCL-W and BCL-XL proteins (Lasica 2021). It is used for treating patients with several haematological malignancies including CLL, AML, small lymphocytic lymphoma and multiple myeloma (MM).

Venetoclax has the following structure:

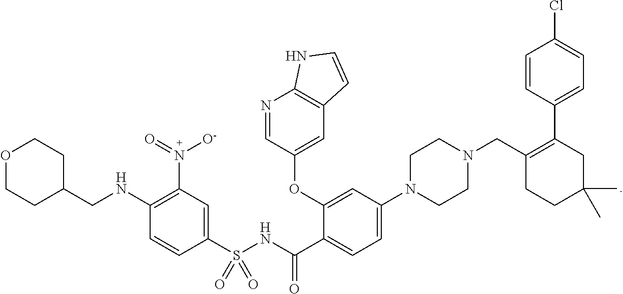

The IUPAC name of Venetoclax is 4-(4-{[2-(4-Chlorophenyl)-4,4-dimethyl-1-cyclohexen-1-yl]methyl}-1-piperazinyl)-N-({3-nitro-4-[(tetrahydro-2H-pyran-4-ylmethyl)amino]phenyl}sulfonyl)-2-(1H-pyrrolo[2,3-b]pyridin-5-yloxy)benzamide.

Navitoclax (also referred as ABT-263) is an oral BCL-2 inhibitor with high affinity toward BCL-2 and BCL-XL proteins, but not to MCL-1 protein (Anuar 2020). Navitoclax treatment has been investigated in patients with several haematological malignancies such as a CLL, DLBCL, MCL, FL, SLL, classic Hodgkin's lymphoma, NK/T-cell lymphoma and MZL. Common toxic effects observed for Navitoclax include gastrointestinal disorders, infection, fatigue thrombocytopenia, lymphocytopenia, and an increase in aminotransferases.

Navitoclax has the following structure:

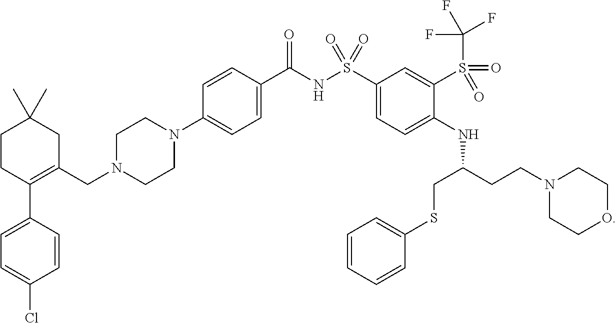 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

Navitoclax has the preferred IUPAC name of 4-(4-{[2-(4-Chlorophenyl)-5,5-dimethylcyclohex-1-en-1-yl]methyl}piperazin-1-yl)-N-(4-{[(2R)-4-(morpholin-4-yl)-1-(phenylsulfanyl)butan-2-yl]amino}-3-(trifluoromethanesulfonyl)benzene-1-sulfonyl)benzamide.

This compound, and methods to make it, are disclosed in U.S. Patent Application Publication No. 2007/0027135 and WO2007/040650 incorporated by reference herein in its entirety. Obatoclax mesylate is an intravenously administered BCL-2 inhibitor binding to several BCL-2 protein family members: BCL-2, BCL-XL, BCL-w, BCL-B, BFL-1, and MCL-1 (Goard 2013). Obatoclax mesylate treatment has been investigated in patients with AML, ALL, CLL, myelodysplasia and myelofibrosis. Neurological symptoms are the most common toxicities associated with obatoclax mesylate. Other relatively common observed toxicities include gastrointestinal symptoms, dyspnoea, and cough.

Obatoclax has the following structure:

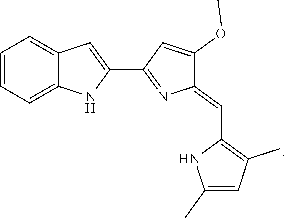 [SMILES: COC1=CC(c2cc3ccccc3[nH]2)=N/C1=C\c1[nH]c(C)cc1C]

The IUPAC name of Obatoclax is 2-(2-((3,5-Dimethyl-1H-pyrrol-2-yl)methylene)-3-methoxy-2H-pyrrol-5-yl)-1H-indole.

Palcitoclax (also referred as APG-1252) is a novel intravenously-administered BCL-2 inhibitor binding to BCL-2 and BCL-XL proteins (Lakhani 2020). It is investigated in patients with metastatic solid tumours. Thrombocytopenia and liver enzyme increases are the most common observed toxicities.

Palcitoclax has the structure:

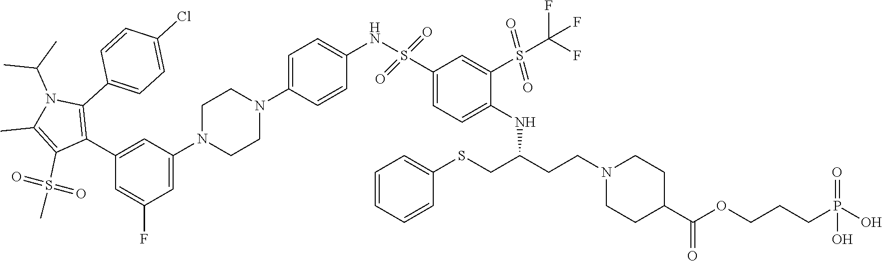 [SMILES: Cc1c(S(C)(=O)=O)c(-c2cc(F)cc(N3CCN(c4ccc(NS(=O)(=O)c5ccc(N[C@H](CCN6CCC(C(=O)OCCCP(=O)(O)O)CC6)CSc6ccccc6)c(S(=O)(=O)C(F)(F)F)c5)cc4)CC3)c2)c(-c2ccc(Cl)cc2)n1C(C)C]

The IUPAC name of Palcitoclax is 3-[1-[(3R)-3-[4-[[4-[4-[3-[2-(4-chlorophenyl)-5-methyl-4-methylsulfonyl-1-propan-2-ylpyrrol-3-yl]-5-fluorophenyl]piperazin-1-yl]phenyl]sulfamoyl]-2-(trifluoromethylsulfonyl)anilino]-4-phenylsulfanylbutyl]piperidine-4-carbonyl]oxypropylphosphonic acid.

Lisaftoclax (also referred as APG-2575) is a novel orally bioavailable BCL-2 inhibitor binding to BCL-2 and BCL-XL (Davids 2021). It is investigated in patients with CLL, SLL and other haematological malignancies (Ailawadhi 2021, NCT05147467). The most common toxicities include neutropenia, anemia, fatigue, diarrhea and nausea.

Lisaftoclax has the following structure:

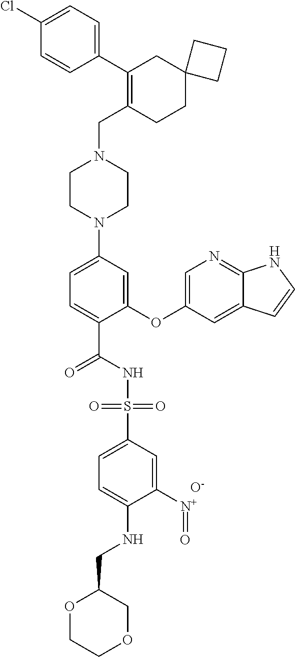

The IUPAC name of Lisaftoclax is (S)—N-((4-(((1,4-dioxan-2-yl)methyl)amino)-3-nitrophenyl)sulfonyl)-2-((1H-pyrrolo[23-b]pyridine-5-yl)oxy)-4-(4-((6-(4-chlorophenyl)spiro[3,5]non-6-en-7-yl)methyl)piperazin-1-yl)benzamide.

Lisaftoclax is compound 6 in WO2018/027097. Its synthesis and characterisation are described in example 6 of WO2018/027097 (pages 87-89).

LP-118 is a novel orally bioavailable BCL-2 inhibitor binding to BCL-2 and BCL-XL (Ravikrishnan 2021). It is investigated in patients with multiple haematological malignancies including non-Hodgkin lymphoma, Richter transformation, MM, T-cell-prolymphocytic Leukemia, AML, ALL, myeodysplastic syndrome, myelodysplastic/myeloproliferative neoplasm, MF, CLL, SLL, chronic myelomonocytic leukemia-2 and myelodysplastic neoplasm in blast phase (NCT04771572).

The structure of LP-118 has not been disclosed yet. It is likely covered by the following Markush structure described in U.S. Pat. No. 10,377,755:

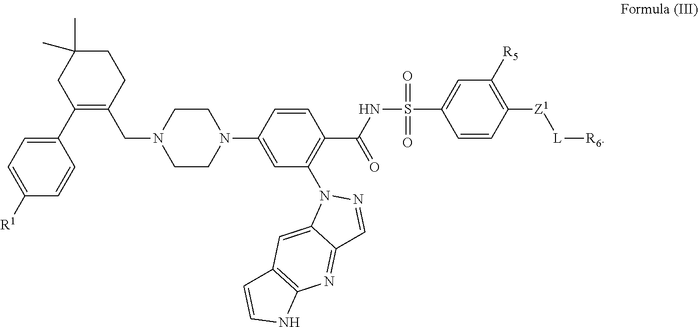

S55746 (also referred as BCL201) is a novel orally bioavailable BCL-2 inhibitor binding to BCL-2. It has been investigated in FL and MCL (NCT02603445).

S55746 has the following structure:

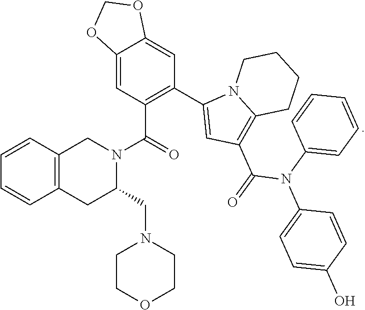 [SMILES: O=C(c1cc(-c2cc3c(cc2C(=O)N2Cc4ccccc4C[C@H]2CN2CCOCC2)OCO3)n2c1CCCC2)N(c1ccccc1)c1ccc(O)cc1]

The IUPAC name of S55746 is (S)—N-(4-hydroxyphenyl)-3-(6-(3-(morpholinomethyl)-1,2,3,4-tetrahydroisoquinoline-2-carbonyl)benzo[d][1,3]dioxol-5-yl)-N-phenyl-5,6,7,8-tetrahydroindolizine-1-carboxamide.

S64315 (also referred as MIK665) is a novel intravenously administered BCL-2 inhibitor binding to MCL-1. It is investigated in patients with AML (NCT04629443).

S64315 has the following structure:

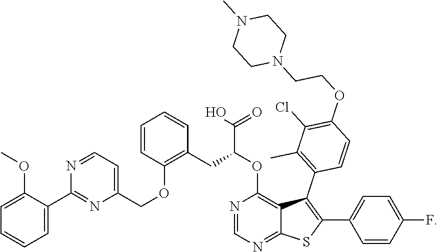

The IUPAC name of S64315 is (R)-2-((5-(3-chloro-2-methyl-4-(2-(4-methylpiperazin-1-yl)ethoxy)phenyl)-6-(4-fluorophenyl)thieno[2,3-d]pyrimidin-4-yl)oxy)-3-(2-((2-(2-methoxyphenyl)pyrimidin-4-yl)methoxy)phenyl)propanoic acid.

S63845 is a small molecule BCL-2 inhibitor specifically binding to MCL-1 (Kotschy 2016). It is investigated in patients with AML (NCT04629443). S63845 potently kills MCL1-dependent cancer cells, including multiple myeloma, leukaemia and lymphoma cells.

S63845 has the structure:

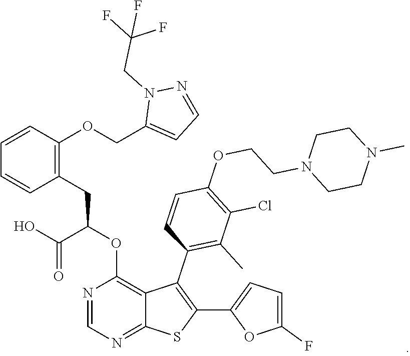

The IUPAC name of S63845 is (R)-2-((5-(3-chloro-2-methyl-4-(2-(4-methylpiperazin-1-yl)ethoxy)phenyl)-6-(5-fluorofuran-2-yl)thieno[2,3-d]pyrimidin-4-yl)oxy)-3-(2-((1-(2,2,2-trifluoroethyl)-1H-pyrazol-5-yl)methoxy)phenyl)propanoic acid.

In some embodiments, the further chemotherapeutic agent may be a second BCL-2 inhibitor.

In other embodiments, the further chemotherapeutic agent may be a DNA methyltransferase inhibitor. DNA methylation mediated by DNA methyltransferase is an important epigenetic process regulating gene expression and plays a key role in silencing tumor suppressor genes in cancer. Hence it has become a promising therapeutic target for cancer treatment especially for haematological tumours (Zhang 2022), DNMT inhibitors can also enhance the immunogenicity of tumour cells by promoting tumour antigen presentation or enhancing the function of cytotoxic T cells. Thus DNMT is also a rational therapeutic target in haematological malignancies. First generation DNMT inhibitors are however characterized by high toxicity, poor selectivity and low bioavailability.

DNMT inhibitors include but are not limited to 5-azacitidine (azacytidine (AZA)), 5-aza-2′-deoxycytidine (decitabine (DAC)), clofarabine, gaudecitabine and GSK3685032. 5-azacitidine (azacytidine (AZA)) has the structure:

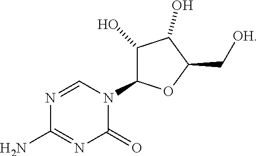 [SMILES: Nc1ncn([C@@H]2O[C@H](CO)[C@@H](O)[C@H]2O)c(=O)n1]

5-Azacitidine is a cytarabine derivative synthesized in 1964, and was first approved by the FDA in 2004.

Decitabine has the structure:

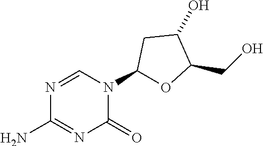 [SMILES: Nc1ncn([C@H]2C[C@H](O)[C@@H](CO)O2)c(=O)n1]

Decitabine was approved by the FDA in 2006. The inhibitory activity of DNMT is 30 times more than that of azacytidine (Zhang 2022).

Clofarabine has the structure:

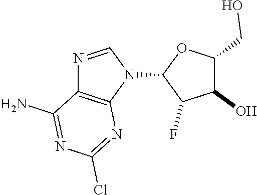 [SMILES: Nc1nc(Cl)nc2c1ncn2[C@@H]1O[C@H](CO)[C@@H](O)[C@@H]1F]

Clofarabine is a purine nucleoside DNMTi, which was approved by the FDA in 2004.

Guadecitabine (SGI-110) has the structure:

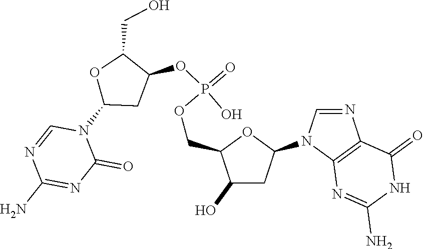 [SMILES: Nc1ncn([C@H]2C[C@H](OP(=O)(O)OC[C@H]3O[C@@H](n4cnc5c(=O)[nH]c(N)nc54)C[C@H]3O)[C@@H](CO)O2)c(=O)n1]

is a dinucleotide derivative of decitabine.

GSK3685032 has the structure:

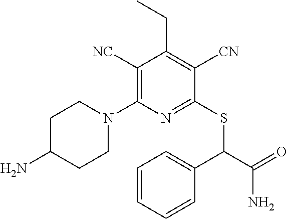 [SMILES: CCc1c(C#N)c(SC(C(N)=O)c2ccccc2)nc(N2CCC(N)CC2)c1C#N]

and is described in Pappalardi 2021. It acts as a competitive inhibitor of DNMT1 via competition with the DNMT1 active-site loop and target-recognition domain to incorporate into the hemi-methylated DNA.

Other DNMT inhibitors include, but are not limited to: RX-3117 (TV-1360), 5-fluoro-2-deoxycytidine, 6-dihydro-5-azacytidine (DHAC), fazarabine, cladribine, fludarabine, procaine, epgallocatechin gallate (EGCG), hydralazine, genistein, equol, curcumin, disulfiram, resveratrol, caffeic acid, CP-4200, zebularine, NPEOC-DAC, T-dCyd, 5-aza-t-dCyd, RG108, DC-05, DC-501, DC-517, SGI-1027, CM-272, CM-579, Psammaplin A, Psammaplin G and UV15008.

### Definitions

The language “pharmaceutical composition” includes compositions comprising an active ingredient and a pharmaceutically acceptable excipient, carrier or diluent, wherein the active ingredient is Compound 1 or a pharmaceutically acceptable salt thereof, or a BCL-2 inhibitor or a pharmaceutically acceptable salt thereof, or the further chemotherapeutic agent. The language “pharmaceutically acceptable excipient, carrier or diluent” includes compounds, materials, compositions, and/or dosage forms which are, within the scope of sound medical judgment, suitable for use in contact with the tissues of human beings and animals without excessive toxicity, irritation, allergic response, or other problem or complication, as ascertained by one of skill in the art. In some embodiments, the pharmaceutical compositions are in solid dosage forms, such as capsules, tablets, granules, powders or sachets. In some embodiments, the pharmaceutical compositions are in the form of a sterile injectable solution in one or more aqueous or non-aqueous non-toxic parenterally acceptable buffer systems, diluents, solubilizing agents, co-solvents, or carriers. A sterile injectable preparation may also be a sterile injectable aqueous or oily suspension or suspension in a non-aqueous diluent, carrier or co-solvent, which may be formulated according to known procedures using one or more of the appropriate dispersing or wetting agents and suspending agents. The pharmaceutical compositions could be a solution for iv bolus/infusion injection or a lyophilized system (either alone or with excipients) for reconstitution with a buffer system with or without other excipients. The lyophilized freeze-dried material may be prepared from non-aqueous solvents or aqueous solvents. The dosage form could also be a concentrate for further dilution for subsequent infusion.

The language “treat,” “treating” and “treatment” includes the reduction or inhibition of tumour cells of a haematological malignancy in a subject, amelioration of one or more symptoms of a haematological malignancy in a subject, or the slowing or delaying of progression of a haematological malignancy in a subject. The language “treat,” “treating” and “treatment” also includes the reduction or inhibition of the growth of a tumor or proliferation of cancerous cells in a subject.

The language “inhibit”, “inhibition” or “inhibiting” includes a decrease in the baseline activity of a biological activity or process.

The term “subject” includes warm-blooded mammals, for example, primates, dogs, cats, rabbits, rats, and mice. In some embodiments, the subject is a primate, for example, a human. In some embodiments, the subject is suffering from a haematological malignancy.

The language “therapeutically effective amount” includes that amount of Compound 1 and that amount of a BCL-2 inhibitor which together will elicit a biological or medical response in a subject, for example, the reduction or inhibition of tumour cells; amelioration of symptoms of a haematological malignancy; or the slowing or delaying of progression of a haematological malignancy. In some embodiments, the language “therapeutically effective amount” includes the amount of Compound 1 and a BCL-2 inhibitor together that is effective to at least partially alleviate, inhibit, and/or ameliorate a haematological malignancy or inhibit tumour cells and/or reduce or inhibit the proliferation of cancerous cells in a subject. In some embodiments, the language “therapeutically effective amount” includes the amount of Compound 1, the BCL-2 inhibitor and the further chemotherapeutic agent together that is effective to at least partially alleviate, inhibit, and/or ameliorate a haematological malignancy or inhibit tumour cells and/or reduce or inhibit the proliferation of cancerous cells in a subject.

In some embodiments, disclosed is a method of treating a haematological malignancy in a subject in need thereof, comprising administering to the subject a first amount of Compound 1 or a pharmaceutically acceptable salt thereof, and a second amount of a BCL-2 inhibitor or a pharmaceutically acceptable salt thereof. In the method, the first amount and the second amount together comprise a therapeutically effective amount. In some of these embodiments, a third amount of a further chemotherapeutic agent is administered.

In some embodiments, disclosed is Compound 1, or a pharmaceutically acceptable salt thereof, for use in the treatment of a haematological malignancy in a subject, wherein said treatment comprises the separate, sequential or simultaneous administration of i) said Compound 1, or a pharmaceutically acceptable salt thereof, and ii) a BCL-2 inhibitor or a pharmaceutically acceptable salt thereof, to said subject. In some of these embodiments, the treatment further comprises the separate, sequential or simultaneous administration of iii) a further chemotherapeutic agent, to said subject.

In some embodiments, disclosed is a BCL-2 inhibitor, or a pharmaceutically acceptable salt thereof, for use in the treatment of a haematological malignancy in a subject, wherein said treatment comprises the separate, sequential or simultaneous administration of i) said BCL-2 inhibitor, or a pharmaceutically acceptable salt thereof, and ii) Compound 1, or a pharmaceutically acceptable salt thereof, to said subject. In some of these embodiments, the treatment further comprises the separate, sequential or simultaneous administration of iii) a further chemotherapeutic agent, to said subject.

In some embodiments, disclosed is the use of Compound 1, or a pharmaceutically acceptable salt thereof, in the manufacture of a medicament for use in the treatment of a haematological malignancy in a subject, wherein said treatment comprises the separate, sequential or simultaneous administration of i) said medicament comprising Compound 1, or a pharmaceutically acceptable salt thereof, and ii) a BCL-2 inhibitor, or a pharmaceutically acceptable salt thereof, to said subject. In some of these embodiments, the treatment further comprises the separate, sequential or simultaneous administration of iii) a further chemotherapeutic agent, to said subject.

In some embodiments, Compound 1 or a pharmaceutically acceptable salt thereof and a BCL-2 inhibitor or a pharmaceutically acceptable salt thereof (and the further chemotherapeutic agent, if present) are administered separately, sequentially or simultaneously in a treatment cycle. In some embodiments, Compound 1 or a pharmaceutically acceptable salt thereof is continuously administered in the treatment cycle and a BCL-2 inhibitor or a pharmaceutically acceptable salt is also continuously administered in the treatment cycle. In some of these embodiments, the further chemotherapeutic agent is also continuously administered in the treatment cycle.

In some embodiments the BCL-2 inhibitor is selected from Venetoclax, Navitoclax, Obatoclax mesylate, Palcitoclax. Lisaftoclax, LP-118, S55746 or S64315. In further embodiments the BCL-2 inhibitor is Venetoclax.

The term “continuous” or “continuously” refers to administration of a therapeutic agent, e.g. Compound 1, at regular intervals without stopping or interruption, i.e., no void day. By “void day”, it is meant a day when a therapeutic agent is not administered.

A “cycle”, “treatment cycle” or “dosing schedule”, as used herein, refers to a period of combination treatment that is repeated on a regular schedule. For example, the treatment can be given for one week, two weeks, or three weeks wherein Compound 1 and a BCL-2 inhibitor are administered in a coordinated fashion. In some embodiments, a treatment cycle is about 1 week to about 3 months. In some embodiments, a treatment cycle is about 5 days to about 1 month. In some embodiments, a treatment cycle is about 1 week to about 3 weeks. In some embodiments, a treatment cycle is about 1 week, about 10 days, about 2 weeks, about 3 weeks, about 4 weeks, about 2 months, or about 3 months.

In some embodiments, Compound 1 or a pharmaceutically acceptable salt thereof and a BCL-2 inhibitor or a pharmaceutically acceptable salt thereof (and the further chemotherapeutic agent, if present) are administered to the human subject in one or more treatment cycles, e.g., a treatment course. A “treatment course” comprises multiple treatment cycles, which can be repeated on a regular schedule, or adjusted as a tapered schedule as the patient's disease progression is monitored. For example, a patient's treatment cycles can have longer periods of treatment and/or shorter periods of rest at the beginning of a treatment course (e.g., when the patient is first diagnosed), and as the cancer enters remission, the rest period lengthens, thereby increasing the length of one treatment cycle. The period of time for treatment and rest in a treatment cycle, the number of treatment cycles, and the length of time for the treatment course can be determined and adjusted throughout the treatment course by the skilled artisan based on the patient's disease progression, treatment tolerance, and prognosis. In some embodiments, the method comprises 1 to 10 treatment cycles. In some embodiments, the method comprises 2 to 8 treatment cycles.

In some embodiments, Compound 1 or a pharmaceutically acceptable salt thereof is administered for 28 days in a 28-day treatment cycle.

### Dosage

In some embodiments, Compound 1 or a pharmaceutically acceptable salt thereof is administered orally. In some embodiments, Compound 1 or a pharmaceutically acceptable salt thereof is in tablet dosage form. In some embodiments, Compound 1 as the hemi-fumarate salt is administered in a dose of 40 mg per day. In some embodiments Compound 1 as the hemi-fumarate salt is administered in a dose of 80 mg per day.

In some embodiments, a BCL-2 inhibitor or a pharmaceutically acceptable salt thereof is administered orally. In some embodiments, a BCL-2 inhibitor or a pharmaceutically acceptable salt thereof is in tablet dosage form. In some embodiments, a BCL-2 inhibitor or a pharmaceutically acceptable salt thereof is in capsule dosage form. In some embodiments, a BCL-2 inhibitor or a pharmaceutically acceptable salt thereof is administered in a dose between 10 mg and 1 g per day.

In some embodiments, a BCL-2 inhibitor or a pharmaceutically acceptable salt thereof is administered as an intravenous (IV) infusion.

In some embodiments, the further chemotherapeutic agent is administered orally. In some embodiments, the further chemotherapeutic agent is in tablet dosage form. In some embodiments, the further chemotherapeutic agent is in capsule dosage form. In some embodiments, the further chemotherapeutic agent is administered in a dose between 10 mg and 1 g per day.

In some embodiments, Compound 1 and a BCL-2 inhibitor are taken together on an empty stomach, with no food two hours before, and one hour after.

In some embodiments, Compound 1 is taken on an empty stomach, with no food two hours before, and one hour after, and the BCL-2 inhibitor is taken with food, so either at least two hours before, or one hour after Compound 1.

In some embodiments, the BCL-2 inhibitor is dosed weekly.

In some embodiments, disclosed is a pharmaceutical product comprising i) Compound 1 or a pharmaceutically acceptable salt thereof, and ii) a BCL-2 inhibitor or a pharmaceutically acceptable salt thereof. In some embodiments, Compound 1 or a pharmaceutically acceptable salt thereof, and a BCL-2 inhibitor or a pharmaceutically acceptable salt thereof are present in a single dosage form. In some embodiments, Compound 1 or a pharmaceutically acceptable salt thereof, and a BCL-2 inhibitor or a pharmaceutically acceptable salt thereof are present separate dosage forms. In some of these embodiments, the pharmaceutical product further comprises iii) a further chemotherapeutic agent.

In some embodiments, disclosed is a kit comprising: a first pharmaceutical composition comprising Compound 1, or a pharmaceutically acceptable salt thereof; a second pharmaceutical composition comprising a BCL-2 inhibitor, or a pharmaceutically acceptable salt thereof; and instructions for using the first and second pharmaceutical compositions in combination. In some of these embodiments, the kit further comprises a third pharmaceutical composition comprising a further chemotherapeutic agent, and the instructions are for using the first, second and third pharmaceutical compositions in combination.

In some embodiments, the BCL-2 inhibitor is selected from Venetoclax, Navitoclax, Obatoclax mesylate, Palcitoclax, Lisaftoclax, LP-118, S55746 or S64315.

The dosing schedule for Venetoclax as a monotherapy, which is taken once daily, depends on the tumour type but typically consist of increasing doses over the first days or weeks of therapy (Juerez-Salcedo 2019). For example, in patients with CLL Venetoclax is started at the 20 mg dose level in the first week and the dose level is increased to 50 mg in the second week, 100 mg in the third week, 200 mg in the fourth week and 400 mg in the fifth week and beyond. In AML Venetoclax is dosing is increased from 100 mg on day one, to 200 mg on day two, to 400 mg on day three and to 400 mg or 600 mg on day four and beyond depending on the combination medication. Dose reductions of Venetoclax are sometimes required to manage Venetoclax-related toxicities such as tumour lysis syndrome and grade 3 neutropenia and/or non-haematological toxicities. Venetoclax tablets come in three strengths (10, 50, and 100 mg) and are advised to be taken with water and a meal at approximately the same time each day.

In some embodiments the BCL-2 inhibitor is Venetoclax or a pharmaceutically acceptable salt thereof. Venetoclax is administered orally, for example as tablets or capsules dosage form. In some embodiments the Venetoclax is administered at a dose of 10 mg to 400 mg per day, 10 mg to 200 mg per day, 20 mg to 400 mg per day or 10 mg to 100 mg per day. In some embodiments the Venetoclax is administered at a dose of 20 mg, 50 mg, 100 mg, 200 mg or 400 mg per day. In some embodiments the Venetoclax is administered at a dose of 20 mg, 50 mg, 100 mg, 200 mg or 400 mg once per day. In some embodiments Venetoclax or a pharmaceutically acceptable salt thereof and is administered at increasing doses over the first weeks of therapy for example, starting at the 20 mg per day in the first week and the dose level is increased to 50 mg per day in the second week, 100 mg per day in the third week, 200 mg per day in the fourth week and 400 mg per day in the fifth week and beyond. In another embodiment Venetoclax or a pharmaceutically acceptable salt thereof and is administered at 100 mg on day one, to 200 mg on day two, to 400 mg on day three and to 400 mg or 600 mg on day four and beyond. In some embodiments Venetoclax or a pharmaceutically acceptable salt thereof and is administered at 400 mg per day.

In some embodiments, disclosed is a pharmaceutical product comprising i) Compound 1, or a pharmaceutically acceptable salt thereof, and ii) Venetoclax, or a pharmaceutically acceptable salt thereof. In some embodiments Compound 1 or a pharmaceutically acceptable salt thereof and Venetoclax or a pharmaceutically acceptable salt thereof are present in a single dosage form. In some embodiments Compound 1 or a pharmaceutically acceptable salt thereof and Venetoclax or a pharmaceutically acceptable salt thereof are present as separate dosage forms.

In some embodiments the BCL-2 inhibitor is Navitoclax. Navitoclax is administered orally for example as a tablet or capsules. The dosing schedule for Navitoclax as a monotherapy, which is taken daily, typically also consists of a dose escalation regimen over one or two weeks reaching a final dose of 250 mg or 325 mg per day. In some embodiments Navitoclax is administered at a dose of 125 to 325 mg per day, or 250 to 325 mg per day. In some embodiments Navitoclax is administered at a dose of 250 mg per day. In some embodiments Navitoclax is administered at a dose of 325 mg per day. In some embodiments, the daily dose of Navitoclax is increased from an initial starting to dose to a final dose level.

In some embodiments, disclosed is a pharmaceutical product comprising i) Compound 1, or a pharmaceutically acceptable salt thereof, and ii) Navitoclax or a pharmaceutically acceptable salt thereof. In some embodiments Compound 1 or a pharmaceutically acceptable salt thereof and Navitoclax or a pharmaceutically acceptable salt thereof are present in a single dosage form. In some embodiments Compound 1 or a pharmaceutically acceptable salt thereof and Navitoclax or a pharmaceutically acceptable salt thereof are present as separate dosage forms.

In some embodiments the BCL-2 inhibitor is Obatoclax mesylate. Obatoclax mesylate is administered intravenously. Obatoclax mesylate infusions are typically given over a 24-hour period and the typical dosing schedule for Obatoclax mesylate infusions consists of 28 mg per square meter body surface area per day for up to four days every two weeks or a set dose of 60 mg every two weeks. In some embodiments the Obatoclax mesylate is administered at a dose of 28 mg per square meter body surface area per day. In some embodiments the Obatoclax mesylate is administered at a dose of 60 mg every two weeks.

In some embodiments the BCL-2 inhibitor is Palcitoclax. Palcitoclax is administered intravenously and its recommended Phase 2 dose is determined to be 240 mg administered once weekly. In some embodiments the Palcitoclax is administered intravenously at a dose of 100 to 300 mg once weekly. In some embodiments, the Palcitoclax is administered at a dose of 120 or 240 mg once weekly.

In some embodiments the BCL-2 inhibitor is Lisaftoclax. Lisaftoclax is administered orally, for example as tablets or capsules. In some embodiments the Lisaftoclax is dosed daily at a dose of 200 to 600 mg, 200 to 400 mg, 300 to 600 mg, 300 mg or 600 mg.

In some embodiments, disclosed is a pharmaceutical product comprising i) Compound 1, or a pharmaceutically acceptable salt thereof, and ii) Lisaftoclax, or a pharmaceutically acceptable salt thereof. In some embodiments Compound 1 or a pharmaceutically acceptable salt thereof and Lisaftoclax or a pharmaceutically acceptable salt thereof are present in a single dosage form. In some embodiments Compound 1 or a pharmaceutically acceptable salt thereof and Lisaftoclax or a pharmaceutically acceptable salt thereof are present as separate dosage forms.

In some embodiments the BCL-2 inhibitor is LP-118. LP-118 is administered orally, for example as tablets or capsules dosage form. In some embodiments the LP-118 is administered at a dose of 10 to 600 mg per day. In some embodiments the LP-118 is administered at a dose of 20 mg per day, 50 mg per day, 100 mg per day, 200 mg per day, 300 mg per day, 400 mg per day, 500 mg per day or 600 mg per day. In some embodiments the LP-118 is administered at a dose of 100 mg per day or 200 mg per day.

In some embodiments the BCL-2 inhibitor is S55746. S55746 is administered orally, for example as tablets or capsules. In some embodiments S55746 is administered at a dose of 50 to 1500 mg per day. In some embodiments S55746 is administered at a dose of up to about 100 mg per day, up to about 200 mg per day, up to about 300 mg per day, up to about 400 mg per day or up to about 500 mg per day. In some embodiments S55746 is administered at a dose of 50 to 1500 mg once per day.

In some embodiments the BCL-2 inhibitor is S64315. S64315 is administered intravenously. In some embodiments the S64315 is administered at a dose of 50 to 250 mg once per week. In some embodiments the S64315 is administered at a dose of up to 100 mg once per week. In some embodiments the S64315 is administered at a dose of up to 200 mg once per week.

In some embodiments the haematological malignancy is lymphoma, including cutaneous B-cell lymphoma, cutaneous T-cell lymphoma, Hodgkin's lymphoma, Non-Hodgkin's lymphoma, FL, mantle cell lymphoma (MCL), MZL, DLBCL, SLL, PTCL and Waldenstrom macroglobulinemia.

In some embodiments the haematological malignancy is leukemia, including ALL, AML, CLL, chronic myelogenous leukemia, hairy cell leukemia, myelodysplastic syndromes, myeloproliferative disorders and myelofibrosis.

In some embodiments the haematological malignancy is myeloma, including multiple myeloma.

Without wishing to be bound by theory, the combination of Compound 1 and a BCL-2 inhibitor may be beneficial for patients not responding or refractory to either Compound 1 or the BCL-2 inhibitor. In addition, the combination of Compound 1 and a BCL-2 inhibitor may deepen the response to either Compound 1 or the BCL-2 inhibitor or make the response more durable. Furthermore, the combination of Compound 1 and a BCL-2 inhibitor may allow for dose reductions of the BCL-2 inhibitor resulting in improved tolerability and quality of life.

Furthermore, where the further chemotherapeutic agent is a second BCL-2 inhibitor, the reasoning above may equally apply. Where the further chemotherapeutic agent is a DNA methyltransferase inhibitor, the combination may be beneficial for patients not responding or refractory to any of Compound 1, the BCL-2 inhibitor and the DNMT inhibitor. In addition, the combination of Compound 1, the BCL-2 inhibitor and the DNMT inhibitor may deepen the response to Compound 1, the BCL-2 inhibitor or the DNMT inhibitor or make the response more durable. Furthermore, the combination of Compound 1, the BCL-2 inhibitor and the DNMT inhibitor may allow for dose reductions of the BCL-2 inhibitor or DNMT inhibitor resulting in improved tolerability and quality of life.

### Methods of the Invention

As described in more detail below, the present inventors have surprisingly found that the combination of Compound 1 with BCL-2 inhibitors has synergistic effects on the proliferation of heamatological malignancy cell lines. Thus, a combination therapy with improved anti-proliferative activity can be provided.

Secondly, the present inventors surprisingly found that the synergistic anti-tumour activity of the combination of Compound 1 with BCL-2 inhibitors is not limited to a specific heamatological malignancy. Thus, a combination therapy with improved therapeutic activity across multiple heamatological malignancies can be provided.

Additionally, the present inventors surprisingly found that the combination of Compound 1 with BCL-2 inhibitors results in tumour cell killing that is not observed with either inhibitor alone. Thus, a combination that can reduce tumour burden can be provided.

Furthermore, the present inventors surprisingly found that the combination of Compound 1 with BCL-2 inhibitors has synergistic effects already at sub-therapeutic doses. Thus, a combination with improved tolerability may be provided.

### EXAMPLES

The compounds of the application will now be further explained by reference to the following non-limiting examples.

### Example 1. Efficacy of Compound 1 Combined with BCL-2 Inhibitor Venetoclax in an In Vitro Assay Using the Cutaneous T-Cell Lymphoma (CTCL) Cell Line HH and the Mantle Cell Lymphoma (MCL) Cell Line SP53

HH (CRL-2105) and SP53 cells were exposed to increasing doses of Compound 1, to increasing doses of Ventoclax, and to increasing doses of the combination of Compound 1 and Venetoclax. Venetoclax was used at a maximum concentration of 10 μM, following an eight-fold dose response design based on 1:4 compound dilutions plus an untreated control. Compound 1 was used at a maximum concentration of 10 μM, following an eight fold dose response design based on 1:3 compound dilutions plus an untreated control.

Cells were incubated for 72 hours at 37° C. and 5% CO₂. The antiproliferative effect of single and combination treatments were determined by adding 20 μL MTT [3-(4,5-dimethylthiazolyl-2)-2, 5-diphenyltetrazoliumbromide]reagent (Sigma Aldrich, Buchs, Switzerland) to each well and the plates were incubated at 37° C. for 4 hours, followed by adding 50 μL of sodium dodecyl sulfate (SDS) (250 μM SDS, 0.21% fuming HCl) lysis buffer. The lysed cells were kept overnight, and then the absorbance was read at 570 nm using the Cytation 3 instrument (BioTek, Winoosku, VT, USA). The effect of the combinations was determined according to the Chou-Talalay Combination Index (CI) (Chou 2008, Chou 2010) and calculated with the Synergy R package (Lee 2007). The effect of the combinations was defined as strongly synergistic for CI values below <0.3, synergistic for CI values between 0.3 and 0.9, additive for CI values between 0.9 and 1.1, and antagonistic for CI values >1.1.

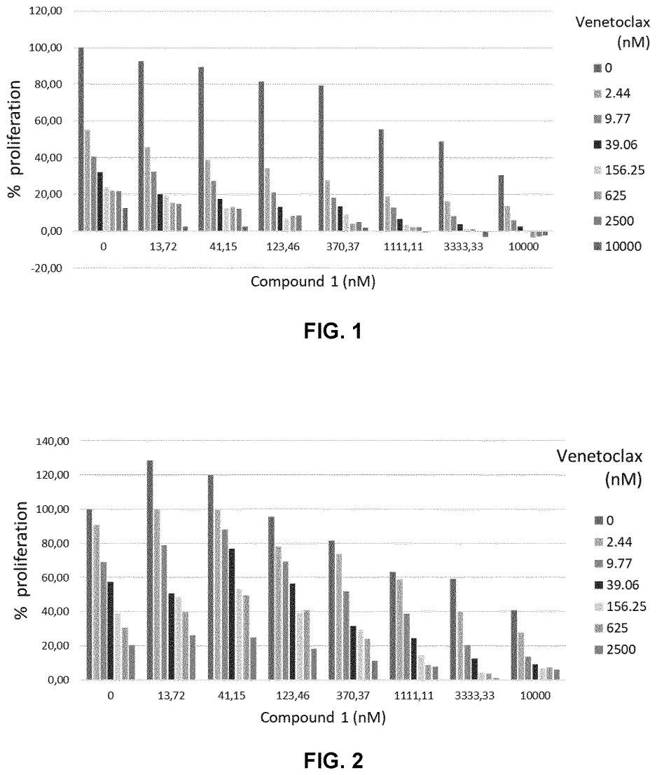

Data from two representative experiments is shown FIGS. 1 and 2.

The combination of Compound 1 and Venetoclax in HH is strongly synergistic with a median CI value of 0.006. The combination of Compound 1 and Venetoclax in SP53 is synergistic with a median CI value of 0.77.

### Example 2. Efficacy of Compound 1 Combined with BCL-2 Inhibitors in an In Vitro Assay Using the Several Different Haematological Cell Lines

Cell lines from different haematological malignancies were exposed to increasing doses of Compound 1, to increasing doses of BCL-2 inhibitor, and to increasing doses of the combination of Compound 1 and BCL-2 inhibitor. Venetoclax, S55746, S64315, and S63845, were used at a maximum concentration of 10 μM, following an eight fold dose response based on 1:4 compound dilutions plus an untreated control. Compound 1 was used at a maximum concentration of 10 μM, following an eight fold dose response based on 1:3 compound dilutions plus an untreated control.

Cells were incubated for 72 hours at 37° C. and 5% CO₂. The antiproliferative effect of single and combination treatments were determined by adding 20 μL MTT [3-(4,5-dimethylthiazolyl-2)-2, 5-diphenyltetrazoliumbromide]reagent (Sigma Aldrich, Buchs, Switzerland) to each well and the plates were incubated at 37° C. for 4 hours, followed by adding 50 μL of sodium dodecyl sulfate (SDS) (250 μM SDS, 0.21% fuming HCl) lysis buffer. The lysed cells were kept overnight, and then the absorbance was read at 570 nm using the Cytation 3 instrument (BioTek, Winoosku, VT, USA). The effect of the combinations was determined according to the Chou-Talalay Combination Index (CI) (Chou 2008, Chou 2010) and calculated with the Synergy R package (Lee 2007). The effect of the combinations was defined as strongly synergistic for CI values below <0.3, synergistic for CI values between 0.3 and 0.9, additive for CI values between 0.9 and 1.1, and antagonistic for CI values >1.1.

CI values from representative experiments is shown in Table 1:

TABLE 1
CI values for the combination of Compound 1 and BCL-2
inhibitors in different haematological cell lines

| | | | 95% CI |
|---|---|---|---|
| BCL-2 | Cell line | CI value | confidence |
| inhibitor | (origin) | (median) | interval |
| S55746 | JVM2 (MCL) | 0.46 | 0.18-0.75 |
| S55746 | TMD8 (DLBCL) | 0.4 | 0.35-0.51 |
| S55746 | MEC1 (CLL) | 0.21 | |
| Venetoclax | Granta519 (MCL) | 0.81 | 0.66-1.0 |
| Venetoclax | JVM2 (MCL) | 0.15 | 0.096-0.21 |
| Venetoclax | MJ (CTCL) | 0.47 | 0.3-0.8 |
| Venetoclax | MEC1 (CLL) | 0.05 | |
| S63845 | Granta519 (MCL) | 0.47 | 0.2-1.4 |
| S63845 | JVM2 (MCL) | 0.33 | 0.26-0.39 |
| S63845 | MJ (CTCL) | 0.67 | 0.41-1.4 |
| S63845 | TMD8 (DLBCL) | 0.86 | 0.43-1.3 |
| S64315 | JVM2 (MCL) | 0.24 | 0.18-0.41 |
| S64315 | SP49 (MCL) | 0.71 | 0.49-1.2 |
| S64315 | TMD8 (CTCL) | 0.23 | 0.093-0.52 |
| S64315 | MEC1 (CLL) | 0.88 | |

### REFERENCES

A number of publications are cited above in order to more fully describe and disclose the invention and the state of the art to which the invention pertains. Full citations for these references are provided below. The entirety of each of these references is incorporated herein

| | Reference | Doi |
|---|---|---|
| Ailawadhi 2021 | Journal of Clinical Oncology | 10.1200/JCO.2021.39.15_suppl.7502 |
| | (2021), 39, no. 15_suppl | |
| Carlo-Stella | Carlo-Stella, C. et al., EHA | |
| 2022 | 2022, poster 1116 | |
| Chou 2008 | Chou, T., Leukemia & | 10.1080/10428190802353591 |
| | Lymphoma (2008), 49(11) | |
| Chou 2010 | Chou, T., Cancer Res (2010), | 10.1158/0008-5472.CAN-09-1947 |
| | 70 (2), 440-446 | |
| Davids 2021 | Davids et al., Blood (2021), 138 | 10.1182/blood-2021-152811 |
| | (Supplement 1): 1554. | |
| Goard 2013 | Goard, C. and Schimmer, A., | 10.2147/ce.s42568 |
| | Core Evidence (2013), 8 15-26 | |
| Hampel 2021 | Hampel, P. et al. Blood Cancer | 10.1038/s41408-021-00443-1 |
| | Journal (2021), Vol. 11: 47 | |
| Haselmayer | Haselmayer, P. et al., Frontiers | 10.3389/fimmu.2014.00233 |
| 2014 | in Immunology (2014), Vol. 5, | |
| | Art. 233, p. 1-15 | |
| Intlekofer and | Intlekofer, M. and Younes, A. | 10.1038/nrclinonc.2014.137 |
| Younes 2014 | Nature reviews Clinical | |
| | Oncology (2014), Vol. 11, p. | |
| | 585-596 | |
| Jephcote 2020 | Jephcote, C. et al., | 10.1186/s12940-020-00582-1 |
| | Environmental Health (2020), | |
| | Vol. 19: 53 | |
| Juárez-Salcedo | Juárez-Salcedo, L. et al., Drugs | 10.7573/dic.212574 |
| 2019 | in Context (2019), 8, 212574 | |
| Kapoor 2020 | Kapoor, I. et al., Cell Death & | 10.1038/s41419-020-03144-y |
| | Disease (2020), Vol. 11: 941 | |
| Katarjian 2021 | Katarjian, H. et al., Blood | 10.1038/s41408-021-00425-3 |
| | Cancer Journal (2021), Vol. | |
| | 11: 41 | |
| Kienle and | Kienle D and Stilgenbauer S, | 10.1080/14656566.2020.1737010 |
| Stilgenbauer | Expert Opin Pharmacother. | |
| 2020 | 2020 June; 21(8): 917-929. | |
| Kotschy 2016 | Kotchy, A. et al., Nature (2016), | 10.1038/nature19830 |
| | 538 (7626), 477-482 | |
| Lakhani 2020 | Lakhani et al., Journal of Clinical | 10.1200/JCO.2020.38.15_suppl.3509 |
| | Oncology (2020), 38, no. | |
| | 15_suppl, 3509 | |
| Lee 2007 | Lee, J. et al., J Biopharm Stat | 10.1080/10543400701199593 |
| | (2007), 17(3), 461-80 | |
| Moujalled 2018 | Moujalled, D. et al., Leukemia | 10.1038/s41375-018-0261-3 |
| | (2019), 33, 905-917 | |
| Pappalardi | Pappalardi, M, et al., Nature | 10.1038/s43018-021-00249-x |
| 2021 | Cancer 2, 1002-1017 (2021) | |
| Perini 2018 | Perini et al. Journal of | 10.1186/s13045-018-0608-2 |
| | Hematology & Oncology (2018) | |
| | 11: 65 | |
| Ploumaki 2023 | Ploumaki, I. et al., Clinical and | 10.1007/s12094-022-03070-9 |
| | Translational Oncology (2023), | |
| | 699 | |
| Ravikrishnan | Ravikrishnan et al., Blood | 10.1182/blood-2021-151852 |
| 2021 | (2021), 138 (Supplement 1): | |
| | 679. | |
| Richardson | Richardson, N. The Lancet | 10.1016/S1470-2045(22)00200-5 |
| 2022 | Oncology (2022), Vol. 23, p. | |
| | 563-566 | |
| Tarantelli 2022 | Tarantelli, C. et al. ENA 2022, | |
| | poster 223 | |
| Zhang 2022 | Zhang, Z. et al. Front Pharmacol | 10.3389/fphar.2022.1072651 |
| | 13, 1072651 (2022) | |

### Statements

1. A method of treating a haematological malignancy in a subject in need thereof, comprising administering to the subject a first amount of a compound of Formula I:

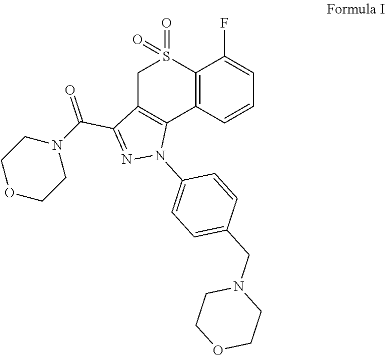 [SMILES: O=C(c1nn(-c2ccc(CN3CCOCC3)cc2)c2c1CS(=O)(=O)c1c(F)cccc1-2)N1CCOCC1]

or a pharmaceutically acceptable salt thereof, and a second amount of a BCL-2 inhibitor or a pharmaceutically acceptable salt thereof, wherein the first amount and the second amount together comprise a therapeutically effective amount.

2. The method according to statement 1, wherein the haematological malignancy is lymphoma, leukaemia, myeloma, myelodysplastic syndrome or a myeloproliferative disease.

3. The method according to statement 2 wherein the lymphoma is cutaneous B-cell lymphoma, cutaneous T-cell lymphoma, Hodgkin's lymphoma, non-Hodgkin's lymphoma, FL, mantle cell lymphoma (MCL), MZL, DLBCL, SLL, PTCL or Waldenstrom macroglobulinemia.

4. The method according to statement 2 wherein the leukaemia is ALL, AML, CLL, chronic myelogenous leukemia, hairy cell leukemia, myelodysplastic syndromes, myeloproliferative disorders and myelofibrosis.

5. The method according to any of statements 1 to 4, wherein the BCL-2 inhibitor is selected from Navitoclax, Obatoclax mesylate, Venetoclax, Lisaftoclax, LP-118, S55746, S64315, or Palcitoclax.

6. The method according to any one of statements 1 to 5, wherein Compound 1 is administered as the hemifumarate salt in a dose of 40 mg or 80 mg per day.

7. The method according to any one of statements 1 to 6, wherein the BCL-2 inhibitor or a pharmaceutically acceptable salt thereof is administered in a dose between 10 mg and 1 g per day.

8. The method according to any one of statements 1 to 7, wherein the BCL-2 inhibitor is Venetoclax, or a pharmaceutically acceptable salt thereof.

9. The method according to any one of statements 1 to 8 wherein the BCL-2 inhibitor is Venetoclax or a pharmaceutically acceptable salt thereof and is administered at a dose of 20 mg, 50 mg, 100 mg, 200 mg or 400 mg per day.

10. A method according to any one of statements 1 to 7, wherein the BCL-2 inhibitor is Navitoclax.

11. A method according to any one of statements 1 to 10, wherein a third amount of a further chemotherapeutic agent is administered.

12. A method according to statement 11, wherein the further chemotherapeutic agent is a second BCL-2 inhibitor.

13. A method according to statement 11, wherein the further chemotherapeutic agent is a DNA methyltransferase inhibitor.

14. A method according to statement 13, wherein the DNA methyltransferase inhibitor is selected from 5-azacitidine, 5-aza-2′-deoxycytidine, clofarabine, gaudecitabine, GSK3685032 RX-3117, 5-fluoro-2-deoxycytidine, 6-dihydro-5-azacytidine, fazarabine, cladribine, fludarabine, procaine, epgallocatechin gallate, hydralazine, genistein, equol, curcumin, disulfiram, resveratrol, caffeic acid, CP-4200, zebularine, NPEOC-DAC, T-dCyd, 5-aza-t-dCyd, RG108, DC-05, DC-501, DC-517, SGI-1027, CM-272, CM-579, Psammaplin A, Psammaplin G and UV15008.

15. A compound of Formula I:

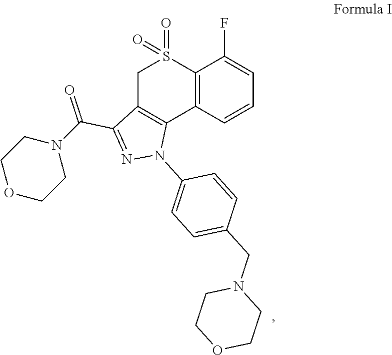 [SMILES: O=C(c1nn(-c2ccc(CN3CCOCC3)cc2)c2c1CS(=O)(=O)c1c(F)cccc1-2)N1CCOCC1]

or a pharmaceutically acceptable salt thereof, for use in the treatment of a haematological malignancy in a subject, wherein said treatment comprises the separate, sequential or simultaneous administration of i) said Compound 1, or a pharmaceutically acceptable salt thereof, and ii) a BCL-2 inhibitor, or a pharmaceutically acceptable salt thereof, to said subject.

16. Compound 1, or a pharmaceutically acceptable salt thereof, for use according to statement 15, wherein the haematological malignancy is lymphoma, leukaemia, myeloma, myelodysplastic syndrome or a myeloproliferative disease.

17. Compound 1, or a pharmaceutically acceptable salt thereof, for use according to statement 16 wherein the lymphoma is cutaneous B-cell lymphoma, cutaneous T-cell lymphoma, Hodgkin's lymphoma, non-Hodgkin's lymphoma, FL, mantle cell lymphoma (MCL), MZL, DLBCL, SLL, PTCL or Waldenstrom macroglobulinemia.

18. Compound 1, or a pharmaceutically acceptable salt thereof, for use according to statement 16 wherein the leukaemia is ALL, AML, CLL, chronic myelogenous leukemia, hairy cell leukemia, myelodysplastic syndromes, myeloproliferative disorders and myelofibrosis.

19. Compound 1, or a pharmaceutically acceptable salt thereof, for use according to any one of statements 15 to 18, wherein the BCL-2 inhibitor is selected from Navitoclax, Obatoclax mesylate, Venetoclax, Lisaftoclax, LP-118, S55746, S64315, or Palcitoclax.

20. Compound 1, or a pharmaceutically acceptable salt thereof, for use according to any one of statements 15 to 19, wherein Compound 1 is administered as the hemifumarate salt in a dose of 40 mg or 80 mg per day.

21. Compound 1, or a pharmaceutically acceptable salt thereof, for use according to any one of statements 15 to 20, wherein the BCL-2 inhibitor or a pharmaceutically acceptable salt thereof is administered in a dose between 10 mg and 1 g per day.

22. Compound 1, or a pharmaceutically acceptable salt thereof, for use according to any one of statements 15 to 21, wherein the BCL-2 inhibitor is Venetoclax, or a pharmaceutically acceptable salt thereof.

23. Compound 1, or a pharmaceutically acceptable salt thereof, for use according to any one of statements 15 to 22 wherein the BCL-2 inhibitor is Venetoclax or a pharmaceutically acceptable salt thereof and is administered at a dose of 20 mg, 50 mg, 100 mg, 200 mg or 400 mg per day.

24. Compound 1, or a pharmaceutically acceptable salt thereof, for use according to any one of statements 15 to 23, wherein the BCL-2 inhibitor is Navitoclax.

25. Compound 1, or a pharmaceutically acceptable salt thereof, for use according to any one of statements 15 to 24, wherein the treatment further comprises the separate, sequential or simultaneous administration of iii) a further chemotherapeutic agent, to said subject.

26. Compound 1, or a pharmaceutically acceptable salt thereof, for use according to statement 25, wherein the further chemotherapeutic agent is a second BCL-2 inhibitor.

27. Compound 1, or a pharmaceutically acceptable salt thereof, for use according to statement 25, wherein the further chemotherapeutic agent is a DNA methyltransferase inhibitor.

28. Compound 1, or a pharmaceutically acceptable salt thereof, for use according to statement 27, wherein the DNA methyltransferase inhibitor is selected from 5-azacitidine, 5-aza-2′-deoxycytidine, clofarabine, gaudecitabine, GSK3685032 RX-3117, 5-fluoro-2-deoxycytidine, 6-dihydro-5-azacytidine, fazarabine, cladribine, fludarabine, procaine, epgallocatechin gallate, hydralazine, genistein, equol, curcumin, disulfiram, resveratrol, caffeic acid, CP-4200, zebularine, NPEOC-DAC, T-dCyd, 5-aza-t-dCyd, RG108, DC-05, DC-501, DC-517, SGI-1027, CM-272, CM-579, Psammaplin A, Psammaplin G and UV15008.

29. A BCL-2 inhibitor, or a pharmaceutically acceptable salt thereof, for use in the treatment of a haematological malignancy in a subject, wherein said treatment comprises the separate, sequential or simultaneous administration of i) said BCL-2 inhibitor or a pharmaceutically acceptable salt thereof, and ii) a compound of Formula I:

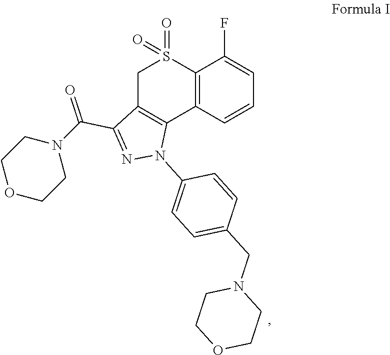 [SMILES: O=C(c1nn(-c2ccc(CN3CCOCC3)cc2)c2c1CS(=O)(=O)c1c(F)cccc1-2)N1CCOCC1]

or a pharmaceutically acceptable salt thereof, to said subject.

30. A BCL-2 inhibitor, or a pharmaceutically acceptable salt thereof, for use according to statement 29, wherein the haematological malignancy is lymphoma, leukaemia, myeloma, myelodysplastic syndrome or a myeloproliferative disease.

31. A BCL-2 inhibitor, or a pharmaceutically acceptable salt thereof, for use according to statement 30 wherein the lymphoma is cutaneous B-cell lymphoma, cutaneous T-cell lymphoma, Hodgkin's lymphoma, non-Hodgkin's lymphoma, FL, mantle cell lymphoma (MCL), MZL, DLBCL, SLL, PTCL or Waldenstrom macroglobulinemia.

32. A BCL-2 inhibitor, or a pharmaceutically acceptable salt thereof, for use according to statement 30 wherein the leukaemia is ALL, AML, CLL, chronic myelogenous leukemia, hairy cell leukemia, myelodysplastic syndromes, myeloproliferative disorders and myelofibrosis.

33. A BCL-2 inhibitor, or a pharmaceutically acceptable salt thereof, for use according to any one of statements 29 to 32, wherein the BCL-2 inhibitor is selected from Navitoclax, Obatoclax mesylate, Venetoclax, Lisaftoclax, LP-118, S55746, S64315, or Palcitoclax.

34. A BCL-2 inhibitor, or a pharmaceutically acceptable salt thereof, for use according to any one of statements 29 to 33, wherein Compound 1 is administered as the hemifumarate salt in a dose of 40 mg or 80 mg per day.

35. A BCL-2 inhibitor, or a pharmaceutically acceptable salt thereof, for use according to any one of statements 29 to 34, wherein the BCL-2 inhibitor or a pharmaceutically acceptable salt thereof is administered in a dose between 10 mg and 1 g per day.

36. A BCL-2 inhibitor, or a pharmaceutically acceptable salt thereof, for use according to any one of statements 29 to 35, wherein the BCL-2 inhibitor is Venetoclax, or a pharmaceutically acceptable salt thereof.

37. A BCL-2 inhibitor, or a pharmaceutically acceptable salt thereof, for use according to any one of statements 29 to 36 wherein the BCL-2 inhibitor is Venetoclax or a pharmaceutically acceptable salt thereof and is administered at a dose of 20 mg, 50 mg, 100 mg, 200 mg or 400 mg per day.

38. A BCL-2 inhibitor, or a pharmaceutically acceptable salt thereof, for use according to any one of statements 29 to 36, wherein the BCL-2 inhibitor is Navitoclax.

39. A BCL-2 inhibitor, or a pharmaceutically acceptable salt thereof, for use according to any one of statements 29 to 38, wherein the treatment further comprises the separate, sequential or simultaneous administration of iii) a further chemotherapeutic agent, to said subject.

40. A BCL-2 inhibitor, or a pharmaceutically acceptable salt thereof, for use according to statement 39, wherein the further chemotherapeutic agent is a second BCL-2 inhibitor.

41. A BCL-2 inhibitor, or a pharmaceutically acceptable salt thereof, for use according to statement 39, wherein the further chemotherapeutic agent is a DNA methyltransferase inhibitor.

42. A BCL-2 inhibitor, or a pharmaceutically acceptable salt thereof, for use according to statement 41, wherein the DNA methyltransferase inhibitor is selected from 5-azacitidine, 5-aza-2′-deoxycytidine, clofarabine, gaudecitabine, GSK3685032 RX-3117, 5-fluoro-2-deoxycytidine, 6-dihydro-5-azacytidine, fazarabine, cladribine, fludarabine, procaine, epgallocatechin gallate, hydralazine, genistein, equol, curcumin, disulfiram, resveratrol, caffeic acid, CP-4200, zebularine, NPEOC-DAC, T-dCyd, 5-aza-t-dCyd, RG108, DC-05, DC-501, DC-517, SGI-1027, CM-272, CM-579, Psammaplin A, Psammaplin G and UV15008.

43. The use of a compound of Formula I:

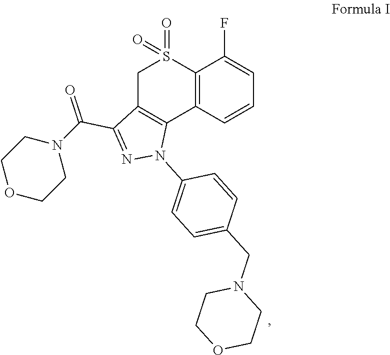 [SMILES: O=C(c1nn(-c2ccc(CN3CCOCC3)cc2)c2c1CS(=O)(=O)c1c(F)cccc1-2)N1CCOCC1]

or a pharmaceutically acceptable salt thereof, in the manufacture of a medicament for use in the treatment of a haematological malignancy, wherein said treatment comprises the separate, sequential or simultaneous administration of i) said medicament comprising Compound 1, or a pharmaceutically acceptable salt thereof, and ii) a BCL-2 inhibitor, or a pharmaceutically acceptable salt thereof, to said subject.

44. The use of Compound 1, or a pharmaceutically acceptable salt thereof, according to statement 43, wherein the haematological malignancy is lymphoma, leukaemia, myeloma, myelodysplastic syndrome or a myeloproliferative disease.

45. The use of Compound 1, or a pharmaceutically acceptable salt thereof, according to statement 44, wherein the lymphoma is cutaneous B-cell lymphoma, cutaneous T-cell lymphoma, Hodgkin's lymphoma, non-Hodgkin's lymphoma, FL, mantle cell lymphoma (MCL), MZL, DLBCL, SLL, PTCL or Waldenstrom macroglobulinemia.

46. The use of Compound 1, or a pharmaceutically acceptable salt thereof, according to statement 44, wherein the leukaemia is ALL, AML, CLL, chronic myelogenous leukemia, hairy cell leukemia, myelodysplastic syndromes, myeloproliferative disorders and myelofibrosis.

47. The use of Compound 1, or a pharmaceutically acceptable salt thereof, according to according to any one of statements 43 to 46, wherein the BCL-2 inhibitor is selected from Navitoclax, Obatoclax mesylate, Venetoclax, Lisaftoclax, LP-118, S55746, S64315, or Palcitoclax.

48. The use of Compound 1, or a pharmaceutically acceptable salt thereof, according to according to any one of statements 43 to 47, wherein Compound 1 is administered as the hemifumarate salt in a dose of 40 mg or 80 mg per day.

49. The use of Compound 1, or a pharmaceutically acceptable salt thereof, according to according to any one of statements 43 to 48, wherein the BCL-2 inhibitor or a pharmaceutically acceptable salt thereof is administered in a dose between 10 mg and 1 g per day.

50. The use of Compound 1, or a pharmaceutically acceptable salt thereof, according to according to any one of statements 43 to 49, wherein the BCL-2 inhibitor is Venetoclax, or a pharmaceutically acceptable salt thereof.

51. The use of Compound 1, or a pharmaceutically acceptable salt thereof, according to according to any one of statements 43 to 50 wherein the BCL-2 inhibitor is Venetoclax or a pharmaceutically acceptable salt thereof and is administered at a dose of 20 mg, 50 mg, 100 mg, 200 mg or 400 mg per day.

52. The use of Compound 1, or a pharmaceutically acceptable salt thereof, according to any one of statements 43 to 49, wherein the BCL-2 inhibitor is Navitoclax.

53. The use of Compound 1, or a pharmaceutically acceptable salt thereof, for use according to any one of statements 43 to 52, wherein the treatment further comprises the separate, sequential or simultaneous administration of iii) a further chemotherapeutic agent, to said subject.

54. The use of Compound 1, or a pharmaceutically acceptable salt thereof, for use according to statement 53, wherein the further chemotherapeutic agent is a second BCL-2 inhibitor.

55. The use of Compound 1, or a pharmaceutically acceptable salt thereof, for use according to statement 53, wherein the further chemotherapeutic agent is a DNA methyltransferase inhibitor.

56. The use of Compound 1, or a pharmaceutically acceptable salt thereof, for use according to statement 55, wherein the DNA methyltransferase inhibitor is selected from 5-azacitidine, 5-aza-2′-deoxycytidine, clofarabine, gaudecitabine, GSK3685032 RX-3117, 5-fluoro-2-deoxycytidine, 6-dihydro-5-azacytidine, fazarabine, cladribine, fludarabine, procaine, epgallocatechin gallate, hydralazine, genistein, equol, curcumin, disulfiram, resveratrol, caffeic acid, CP-4200, zebularine, NPEOC-DAC, T-dCyd, 5-aza-t-dCyd, RG108, DC-05, DC-501, DC-517, SGI-1027, CM-272, CM-579, Psammaplin A, Psammaplin G and UV15008.

57. A pharmaceutical product comprising i) a compound of Formula I:

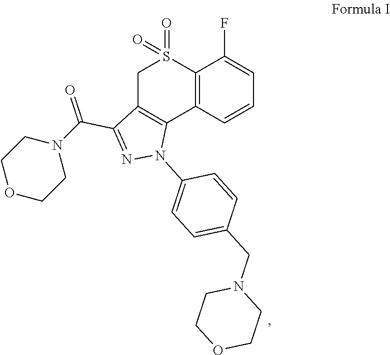 [SMILES: O=C(c1nn(-c2ccc(CN3CCOCC3)cc2)c2c1CS(=O)(=O)c1c(F)cccc1-2)N1CCOCC1]

or a pharmaceutically acceptable salt thereof, and ii) a BCL-2 inhibitor or a pharmaceutically acceptable salt thereof.

58. A pharmaceutical product according to statement 57, wherein the pharmaceutical product further comprises iii) a further chemotherapeutic agent.

59. A kit comprising: a first pharmaceutical composition comprising a compound of Formula I:

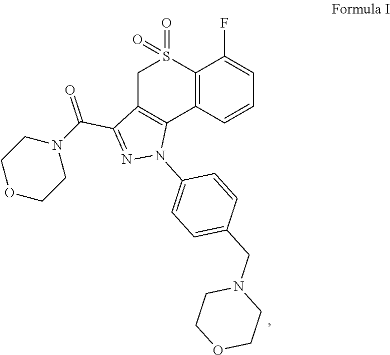 [SMILES: O=C(c1nn(-c2ccc(CN3CCOCC3)cc2)c2c1CS(=O)(=O)c1c(F)cccc1-2)N1CCOCC1]

or a pharmaceutically acceptable salt thereof; a second pharmaceutical composition comprising a BCL-2 inhibitor, or a pharmaceutically acceptable salt thereof; and instructions for using the first and second pharmaceutical compositions in combination.

60. A kit according to statement 59, wherein the kit further comprises a third pharmaceutical composition comprising a further chemotherapeutic agent, and the instructions are for using the first, second and third pharmaceutical compositions in combination.

1-26. (canceled)

27. A method of treating a haematological malignancy in a subject in need thereof, comprising administering to the subject a first amount of a compound of Formula I: 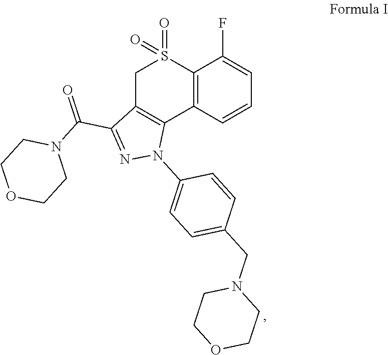 [SMILES: O=C(c1nn(-c2ccc(CN3CCOCC3)cc2)c2c1CS(=O)(=O)c1c(F)cccc1-2)N1CCOCC1]
or a pharmaceutically acceptable salt thereof, and a second amount of a BCL-2 inhibitor, or a pharmaceutically acceptable salt thereof, wherein the first amount and the second amount together comprise a therapeutically effective amount,
wherein the BCL-2 inhibitor is selected from Venetoclax, S55746, S64315, and S63845.

28. The method according to claim 27, wherein the haematological malignancy is lymphoma, leukaemia, myeloma, myelodysplastic syndrome or a myeloproliferative disease.

29. The method according to claim 28, wherein the lymphoma is cutaneous B-cell lymphoma, cutaneous T-cell lymphoma, Hodgkin's lymphoma, non-Hodgkin's lymphoma, FL, mantle cell lymphoma (MCL), MZL, DLBCL, SLL, PTCL or Waldenstrom macroglobulinemia.

30. The method according to claim 28, wherein the leukaemia is ALL, AML, CLL, chronic myelogenous leukemia, hairy cell leukemia, myelodysplastic syndromes, myeloproliferative disorders and myelofibrosis.

31. The method according to claim 27, wherein Compound 1 is administered as a hemifumarate salt in a dose of 40 mg or 80 mg per day.

32. The method according to claim 27, wherein the BCL-2 inhibitor or a pharmaceutically acceptable salt thereof is administered in a dose between 10 mg and 1 g per day.

33. The method according to claim 27, wherein the BCL-2 inhibitor is Venetoclax, or a pharmaceutically acceptable salt thereof.

34. The method according to claim 27, wherein the BCL-2 inhibitor is Venetoclax or a pharmaceutically acceptable salt thereof and is administered at a dose of 20 mg, 50 mg, 100 mg, 200 mg or 400 mg per day.

35. The method according to claim 27, wherein the treatment further comprises the separate, sequential or simultaneous administration of iii) a further chemotherapeutic agent, to said subject, where the further chemotherapeutic agent is a second BCL-2 inhibitor or a DNA methyltransferase inhibitor.

36. A pharmaceutical product comprising i) a compound of Formula I: 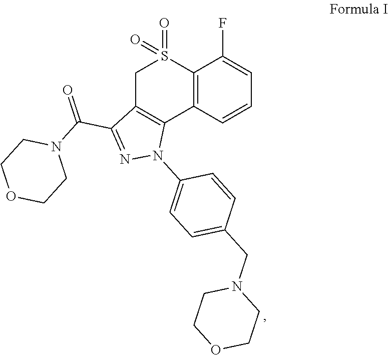 [SMILES: O=C(c1nn(-c2ccc(CN3CCOCC3)cc2)c2c1CS(=O)(=O)c1c(F)cccc1-2)N1CCOCC1]
or a pharmaceutically acceptable salt thereof, and ii) a BCL-2 inhibitor or a pharmaceutically acceptable salt thereof,
wherein the BCL-2 inhibitor is selected from Venetoclax, S55746, S64315, and S63845.

37. The pharmaceutical product according to claim 36, wherein the pharmaceutical product further comprises iii) a further chemotherapeutic agent.

38. A kit comprising: a first pharmaceutical composition comprising a compound of Formula I: 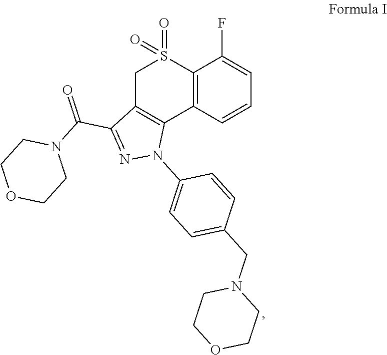 [SMILES: O=C(c1nn(-c2ccc(CN3CCOCC3)cc2)c2c1CS(=O)(=O)c1c(F)cccc1-2)N1CCOCC1]
or a pharmaceutically acceptable salt thereof; a second pharmaceutical composition comprising a BCL-2 inhibitor, or a pharmaceutically acceptable salt thereof; and instructions for using the first and second pharmaceutical compositions in combination,
wherein the BCL-2 inhibitor is selected from Venetoclax, S55746, S64315, and S63845.

39. The kit according to claim 38, wherein the kit further comprises a third pharmaceutical composition comprising a further chemotherapeutic agent, and the instructions are for using the first, second and third pharmaceutical compositions in combination.
